(19) United States
(12) Patent Application Publication
(10) Pub. No.: US 2026/0262994 A1
(43) Pub. Date: Sep. 10, 2026

(54) SYSTEM AND METHOD TO MONITOR NEURAL INTEGRITY

(71) Applicant: Medtronic Xomed LLC, Minneapolis, MN (US)

(72) Inventor: Dongming Shen, Shanghai (CN)

(21) Appl. No.: 19/165,386

(22) PCT Filed: Mar. 21, 2023
(86) PCT No.: PCT/CN2023/082687

§ 371 Date: Sep. 15, 2025

(51) Int. Cl. A61B 5/388; A61B 5/00; A61B 5/294; A61M 16/04

(52) U.S. Cl. CPC A61B 5/388; A61B 5/294; A61B 5/6853; A61B 2505/05; A61M 16/0434

(57) ABSTRACT

A device or system and related methods are disclosed to stimulate and/or monitor a stimulation and/or an evoked response of at least one nerve. The device may be positioned near a subject. A stimulation electrode assembly configured to be positioned relative to a subject is disclosed.

### FIELD

The present disclosure relates to system and method for activity monitoring, and particularly to neural stimulation monitoring devices and methods.

### BACKGROUND

This section provides background information related to the present disclosure which is not necessarily prior art.

During various procedures, such as various throat procedures or other procedures occurring near and/or adjacent to nerve fiber, a determination of nerve integrity or stimulation may be selected. Determining nerve integrity may include ensuring or monitoring stimulation activity along a nerve. This may include transmission of or receiving an induced signal on a nerve. In performing such integrity monitoring, an electrode or electrode containing element is connected to a nerve or nerve fiber to monitor or stimulate the nerve fiber. Monitoring of an induced signal at a single time or over a period of time can assist in determining integrity of a nerve. Various monitoring systems include the NIM-Response® 3.0 sold by Medtronic, Inc. having a place of business in Minneapolis, Minnesota. The monitor systems can include or be operated with an electrode including an APS® electrode that allows for automatic and periodic stimulation of a nerve that may be monitored by the system.

### SUMMARY

This section provides a general summary of the disclosure, and is not a comprehensive disclosure of its full scope or all of its features.

A system to sense provision of stimulation to a selected portion of a subject, such as nerve bundles or paths is disclosed. Selected systems include NIM-Response® 3.0 sold by Medtronic, Inc. that may include a selected cuff or selector for connection of electrodes to nerve modules in a wired or wireless manner. Further, a wireless stimulator assembly can be positioned adjacent to or near a nerve for stimulating the nerve and/or detecting a stimulation of the nerve. The electrodes may include an active fixation that positively connect or surround at least a portion of the nerve bundle. In the alternative, the system may provide a contact electrode that contacts the nerve and is held in place by friction or compression of surrounding tissue.

Various portions can be used to sense an evoked response in a subject. The portions may include one or more electrodes on an endotracheal (ET) tube. The ET tube may include, in addition to the electrodes, holding portions, pressure sensing portions, etc. These portions may be assist holding and/or confirming operation of the ET tube and related sensing system.

A stimulation project may be used to stimulate a selected portion of the subject. The stimulation may be to evoke a response in the subject, such as one that may be measured with a selected system, as discussed herein. The stimulation probe may include a flexible shaft or a portion of a shaft that is flexible to allow placement of a tip at a selected position that is not only along a line from a handle.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of a monitoring system and an electrode assembly;

FIG. 2 is an environmental view of an endotracheal tube, according to various embodiments;

FIG. 3 is a perspective view of a stimulation probe, according to various embodiments;

FIG. 4 is a detailed review of a flexible member portion of the stimulation probe, according to various embodiments;

FIG. 4A is a detail view of a tip end of the flexible member taken in circle 4A of FIG. 4;

FIG. 4B is a detail view of a proximal end of the flexible member taken in circle 4B of FIG. 4;

FIG. 5 is a detail view of a tip end of the flexible member taken in circle 5 of FIG. 3;

FIG. 6 is a detail view of a tip end of the flexible member taken in circle 6 of FIG. 3;

FIG. 7 is a cross-sectional view of the simulation probe taken along lines 7-7 of FIG. 3;

FIG. 7A is a detail view of a tip end of the flexible member taken in circle 7A of FIG. 7;

FIG. 7B is a detail view taken in circle 7B of FIG. 7;

FIG. 7′ is a cross-sectional view of a stimulation probe, according to various embodiments, taken along lines 7′-7′ in FIG. 3;

FIG. 7′A is a detail view of a tip end of the flexible member taken in circle 7′A of FIG. 7′;

FIG. 8 is an illustration of the stimulation probe, according to various embodiments, in a first configuration;

FIG. 9 is an illustration of the stimulation probe, according to various embodiments, in a second configuration; and

FIG. 10 is an illustration of the stimulation probe, according to various embodiments, in a third configuration.

Corresponding reference numerals indicate corresponding parts throughout the several views of the drawings.

### DETAILED DESCRIPTION

Example embodiments will now be described more fully with reference to the accompanying drawings.

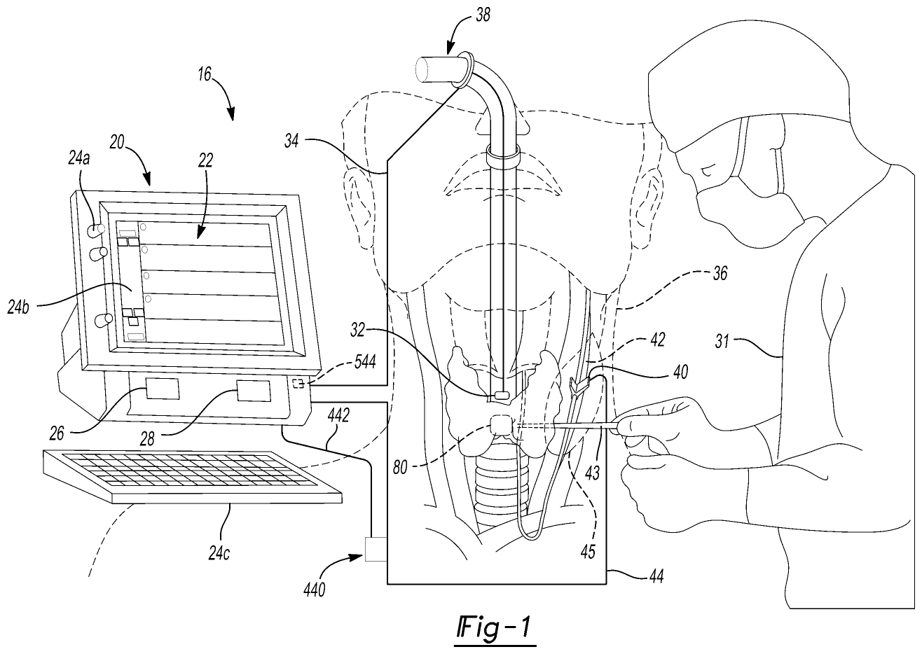

With initial reference to FIG. 1, a monitoring system 16, such as one or more nerve integrity monitoring systems, including one or more of NIM®, NIM Vital®, NIM RESPONSE®, and/or NIM-NEURO® nerve integrity monitor and component parts, may be used during a selected procedure. The monitoring system 16 may include a monitor assembly 20 that has a display screen or device 22 and one or more input devices. The monitoring system 16 may also include monitoring systems such as those disclosed in U.S. patent Ser. No. 14/678,452, filed on Apr. 3, 2015, published as U.S. Pat. App. No. 2016/0287112; U.S. Pat. Nos. 10,039,915; 9,955,882; and 10,799,152, all incorporated herein by reference. The input device may include one or more systems or structures to input commands of information such as knobs 24a, a touch screen 24b, a keyboard 24c, or other appropriate input devices. Input devices may also include audio or other tactile input devices.

The monitor assembly 20 may further include a processor 26 and a memory 28. It is understood that the processor 26 may access the memory 28 to execute instructions stored thereon or access other data on the memory 28. The memory 28 may include a physical memory, such as a spinning hard disk drive, solid state memory, or other appropriate types of memory. Further, the memory 28 may not be incorporated into the monitor assembly 20, but may be accessed by processor 26, such as via a communications network. The processor 26 may be any appropriate processor, as discussed herein, including a general-purpose processor that is operable to execute instructions for generating a selected output, as discussed further herein. The processor 26 may further include onboard memory. Accordingly, the processor 26 may execute instructions stored on memory 28, which may be a non-transitory memory, to provide an output for display on the display device 22. A user 31 may then view the display device 22 for selected purposes, as discussed further herein.

Connected with the monitor assembly 20, may be one or more stimulation or monitoring assemblies. For example, in various procedures such as a thyroidectomy or other thyroid surgeries, monitoring of a recurrent laryngeal nerve (RLN), a vagus nerve, or other appropriate nerve, in a patient 36 may be selected. Monitoring of the RLN may include a nerve monitoring esophageal tube also referred to as an endotracheal (ET) tube 38, which may have one or more conductive electrodes 32 that are in contact with selected portions of the patient 36, such as a human patient. The electrode 32 can be connected to the monitor 20, via a connection 34. It is understood, however, that the connection to the monitor 20 may also be a wireless connection where the monitor 20 receives a wireless transmitted signal from the electrode 32. It is understood, however, that various non-human patient subjects may be monitored. Non-human subjects may include robotic systems, airframes, etc.

In addition, other instruments may be connected to the monitor 20, such as electrode assemblies, including an electrode that may send or receive periodic stimulation pulses, including, according to various embodiments, a connected cuff electrode assembly 40. Other selected or appropriate electrodes include those as disclosed in U.S. Pat. Nos. 9,955,882 and 9,918,669, all incorporated herein by reference.

The stimulation electrode assemblies may be connected with a physical connection, such as a wire 44 to the monitor 20. The connection to the monitor may be wired and/or wireless, such as disclosed in U.S. Pat. No. 9,918,669, incorporated herein by reference. Thus, the monitor may be positioned at any appropriate position relative to the subject 36 and/or the user 31. The monitory 20 may be in a sterile or non-sterile location.

Other instruments may also be connected with the monitor 20 that may be used to send or receive stimulation signals to the patient to assist in determining whether nerve damage or other tissue damage has occurred or could occur. According to various embodiments, a stimulating probe 43 may be manipulated by the user 31, such as a human surgeon, and need not be directly connected to the monitor 20. The stimulating probe, however, may be connected to the monitor 20. The stimulating probe 43 may be used during and/or after making an incision or resection of a portion of the subject, such as forming an incision 45 and/or any appropriately manipulation (e.g., cut, dissection) or incision. The monitor 20 may be provided to monitor signals through or from the electrode assemblies 32, 40, 43 without requiring interactive stimulation or monitoring through the scalpel or other selected instruments performing a procedure on the patient 36. Nevertheless, the stimulating probe 43 may be actively moved by the user to test various portions, such as the nerve 42. According to various embodiments, electrode assemblies may be connected to one or more nerves 42 to generate a stimulation to the nerve 42 at a selected rate. The rate may be selected to account for a refractory period of the nerve. Thus, a pause or period between stimulations may be selected to account for the refractory period.

The operation of the monitoring system and the use of the monitoring system 16 may be similar to the various versions and/or types of the NIM® monitoring system sold by Medtronic, Inc., including the NIM-Response® 3.0 and/or NIM VITAL® nerve monitoring system. In operation, the electrode assembly 40 may be connected with a nerve 42, as discussed further herein, and a signal may be transmitted along the connection 44 from the monitor system 20. The electrode 32 may be used to receive a signal that is transmitted through the nerve 42.

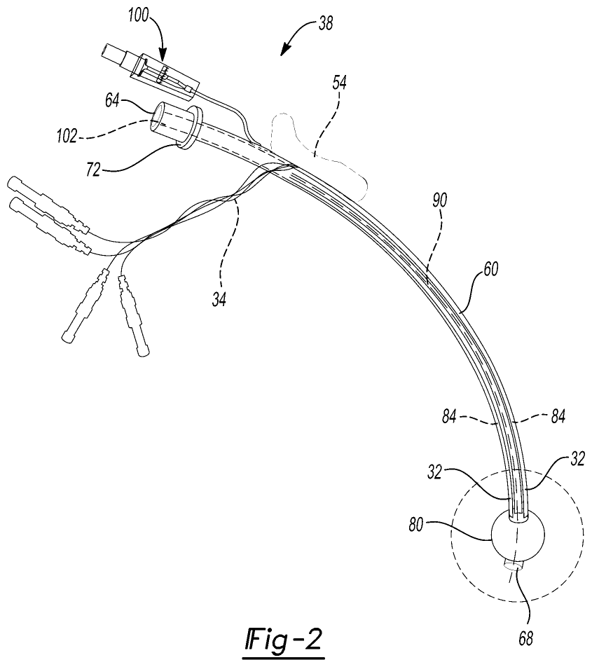

Turning reference to FIG. 2, connected with the monitor assembly 20, may be one or more stimulation or monitoring assemblies. For example, the ET tube assembly 38 that may have one or more monitoring portions, including one or more conductive electrode contacts 32. The electrode contacts 32 may be in contact with selected portions of the patient 36. The electrode contacts 32 may be connected to the monitor 20, via a connection, such as an optional wired connection (also referred to as a line or hardline) 34 or wireless connection including a wireless transmitter 54. It is understood, however, regardless of the connection to the monitor 20, a transmitted signal from the electrode contacts 32 may be made to the monitor assembly 20. An exemplary endotracheal tube may include a NIM Trivantage® monitoring tube sold by Medtronic, Inc. Exemplary endotracheal tubes may further include those disclosed in U.S. Pat. No. 9,918,669, incorporated herein by reference. It is understood, however, that the tube 38 may include portions in addition to those currently available or different from those currently available on the NIM Trivantage® monitoring tube and those discussed above.

The tube 38 may be an EMG endotracheal tube assembly 38 and a corresponding housing 60. The EMG endotracheal tube assembly 38 may include the housing 60 and an electronic assembly including the wireless transmission assembly 54 and/or the wired connection 34. The tube assembly 38 includes a tube proximal (first) end 64 and a distal (second) end 68. The distal end 64 is connected to and/or includes a connector 72, which may be connected to a pump for supplying a selected material such as a gas and/or a fluid to a patient via the tube 38. The tube 38 may be inserted in a throat of the patient 36 and the gas and/or fluid may be supplied to, for example, lungs of the patient 36.

The distal end 68 includes an inflatable portion 80 (shown in an inflated state), which may be used to seal off, for example, a trachea, to prevent any other fluid or substance from passing around the inflated portion 80 and entering the lungs. The inflatable portion 80, as discussed herein, may be inflated in a selected manner. Further, the inflation status of the inflatable portion 80 may be monitored at or near the monitor 20.

The tube assembly 38 includes the contacts 32. The contacts 32 may be in electrical connection with connections 84. The connections may be traces, wires, etc. that may be provided with and/or formed on or in the housing 60. The contacts 32 and/or the electrodes 84 may be painted or printed on and/or relative to the tube housing 60. For example, a conductive paint or ink may be applied to the tube housing 60, or a portion thereof to form the conductive portions 84. Further, the electrodes and transmission portions may include traces that are formed on flexible printed circuit boards and provided relative to the housing 60. A coating or protective layer may also be provided over the painted portions, while allowing the contacts 84 to be exposed to an external environment at selected locations or portions.

The connectors 84 may extend from the contacts 32 to the connection portion, including the wireless transmitter 54 and/or the wired connection 34. The connectors 84 may extend in parallel along the tube housing 60 and are separated as to not be in contact with each other. One or more insulation layers 90 may be applied over the connectors 84 to prevent external electrical contact with the connectors 84. Each of the insulation layers 90 may cover one or more of the connectors 84. The insulation layers 90 may be nonconductive stamps formed of nonconductive material (e.g., rubber).

As discussed above, the tube assembly 38 may be used with the monitoring assembly 20 to monitor an electrical activity signal in the patient 36. A stimulation being sensed through the contacts 32 may be provided through the electrode assembly 40 in connection with the monitor assembly 20. As discussed above, the NIM® stimulation and monitoring system sold by Medtronic, Inc. may be provided to sense at or stimulate the nerve. Upon sensing the stimulation from the nerve (including, for example, an EMG response of a muscle and/or electrical activity related thereto) such as due to placement of the stimulating probe 43 at or adjacent the nerve, the monitoring system 20 may monitor the EMG signal after determining a baseline and to determine the baseline. Further, the user or other appropriate individual may observe the monitoring system 20, such as the display 22, to ensure integrity of nerves during a surgical procedure. Procedures may include a throat or thyroid removal procedure, as discussed above.

The one or multiple contacts 32 can be provided on, such as on a surface at one or more radial positions around the tube assembly 38 for various purposes. In various embodiments, the multiple electrodes and/or contacts therefore may be used for differentiating between left and right nerves, differentiating between different nerves and nerve branches, compensating for users placing the tube contacts in a variety of depths and/or axial positions relative to the anatomy. The multiple electrodes may be placed axially along the tube to allow for measurements at a distance from a selected location as well. Referential recording electrodes may also be placed a distance from other recording electrodes to minimize noise and interference. Upon movement of the contacts 32 relative to the patient 36 (including internal tissue, such as muscle that may include vocal fold in a human larynx), the signal to the monitoring system 20 may change. Change of the signal to the monitoring system 20 may be interpreted or possibly interpreted as an injury to the nerve 56. The system 20, upon determining a change or sensing a change in the received stimulation, may provide an indication to the user 30 that an injury has occurred and that the procedure should be stopped. If the signal to the monitoring system 20, however, changes only due to movement, whether intentional or unintentional, of the tube assembly 40 then no injury has occurred, as disclosed in U.S. Pat. No. 10,799,152, incorporated herein by reference.

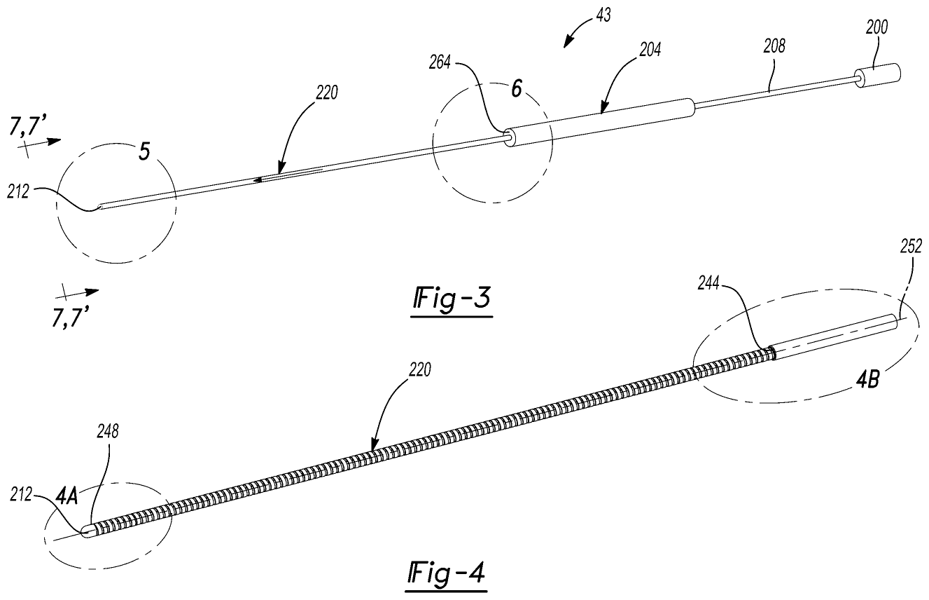

With continuing reference to FIG. 1 and FIG. 2 and additional reference to FIGS. 3, 4, 4A, 4B, 5, and 6, the stimulating probe 43 according to various embodiments is illustrated and described in greater detail. The stimulating probe of 43 may be provided according to various embodiments, and according to an embodiment, as illustrated in FIG. 3, includes a connector or terminal 200 that may be connected to a handle portion 204 via a connector wire or member 208. The connector wire 208 may transmit a stimulation signal from the connector 200 to a stimulating tip 212 of the stimulating probe 43. The connector wire 208 may be a conductive material of any appropriate type such as a metal wire, a conductive polymer, or the like. The connector 200 may be connected to the console 16. In various embodiments, however, the connector 200 may be or may be connected to a wireless member and/or may incorporate a wireless portion or system to provide a simulation signal to the tip 212 in an appropriate manner. As is understood by one skilled in the art, the nerve integrity monitoring system 16 may indicate or control a stimulation signal that is provided through the stimulation tip 212 to the subject 36. The various sensors or conductors may then sense the signal in the patient 36 and the NIM system 16 may monitor the signal to evaluate an integrity of the selected nerve.

The stimulating probe 43 may include the handle 204 that may allow for the user 31 to manipulate the probe 43. As illustrated in FIG. 1, the probe 43 may be positioned relative to a portion of the patient 36. The stimulating probe 43 may then be activated or operated to stimulate a selected portion of the patient 36, such as through the tip 212. The tip 212 may be connected to the handle 204 by a flexible portion 220. The flexible portion 220 may be surrounded by a selected insulator or insulating material 224. Thus, stimulation may be provided to the subject 36 at the tip 212.

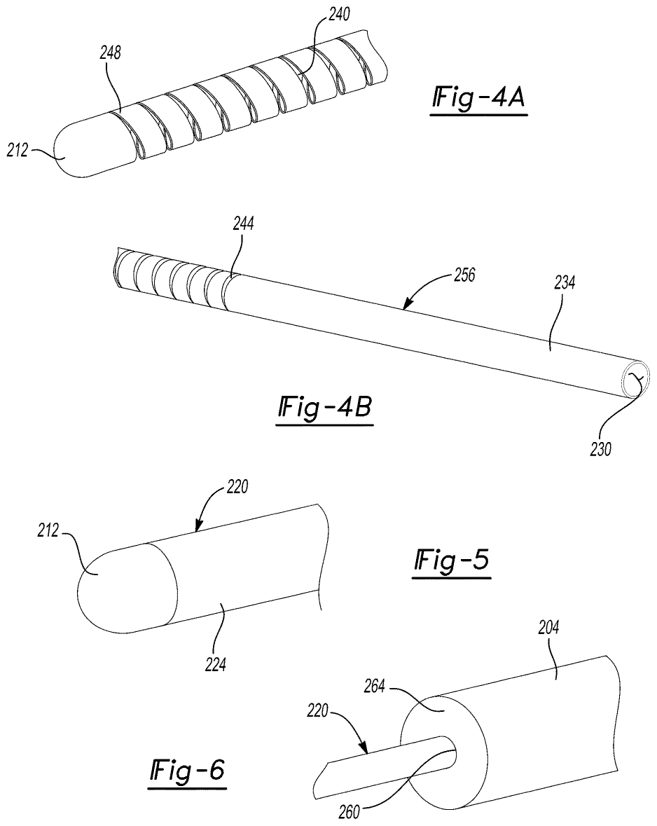

The flexible member 220, surrounded by the insulating material 224, may be formed in an appropriate manner. As illustrated in FIGS. 4, 4A, and 4B the flexible member 220 may be formed as an elongated member that includes a bore or a blind bore 230. Therefore, the flexible number 220 may include an outer wall 234.

The outer wall 234 may be formed of an appropriate material. The outer wall 234, for example, may be formed of an electrically conductive material. The electrically conductive material may include a metal, metal alloy, conductive polymer, carbon, or other appropriate conductive material. The outer wall 234, however may have a selected rigidity.

The flexible member 220 may be made flexible, however, by the outer wall 234 including or having a groove or passage formed therein. For example, a spiral or helical cut 240 may be formed in the outer wall. The spiral cut 240 may extend from a first end 244 to a second end 248. When referred to herein, the spiral cut 240 may refer to a groove in, slit through, or separation of at least a portion of the wall 234. Thus, the spiral cut 240 may refer to a cut entirely through the wall 234 and/or a depression (e.g., groove) therein. Further, the spiral cut 240 may be formed as and/or refer to a spiral or helical manner cut, groove, or slit around (e.g., 360 degrees around) a long axis 252 of the flexible member 220. The spiral cut 240 may be a substantially continuous cut from the first end 244 to the second end 248. In various embodiments, however, the spiral cut 240 may be discontinuous along the length of the flexible member 220 include cut and un-cut portions. Thus, the flexible member may include portions along its length that are more flexible than others. Further, the cut 240 need not be through the outer wall 2340. For example, the cut 240 may be a groove or depression formed in the wall 234 to provide a selected flexibility to the member 220. The spiral cut 240 may allow the flexible member 220 to flex between the first end 244 and the second end 248.

A portion of the flexible number 220 may allow for a connection and may not include the cut 240, but rather may include a substantially rigid portion 256. The rigid portion 256 may be received within the handle 204. As illustrated in FIG. 6, the generally rigid portion 256 may be received within an opening or passage 260 of the handle 204. The passage 260 may be formed in a face end 264 of the handle 204. Accordingly, the flexible number 220 may be formed of a substantially rigid material, such as a metal or metal alloy, that is then formed to include a flexible portion or region 242. The flexible region 242 may be formed by the spiral cut 240 formed in the outer wall 234 of the flexible number 220. The flexible member 220, however, may include a substantially nonflexible portion or rigid portion 256, as noted above.

In various embodiments, the flexible member 220, may be formed of a material that is molded to include the spiral cut 240. Thus, the spiral cut 240 may not be a cut that is formed in a solid member, but may be included due to a manufacture of the flexible member 220. For example, the flexible member may be a molded polymer include the spiral cut 240. Further, the flexible member 220 may be formed as a member that is helical wound around a the central axis 252. If a polymer is used a conductor may be included in the polymer and/or the polymer may be conductive.

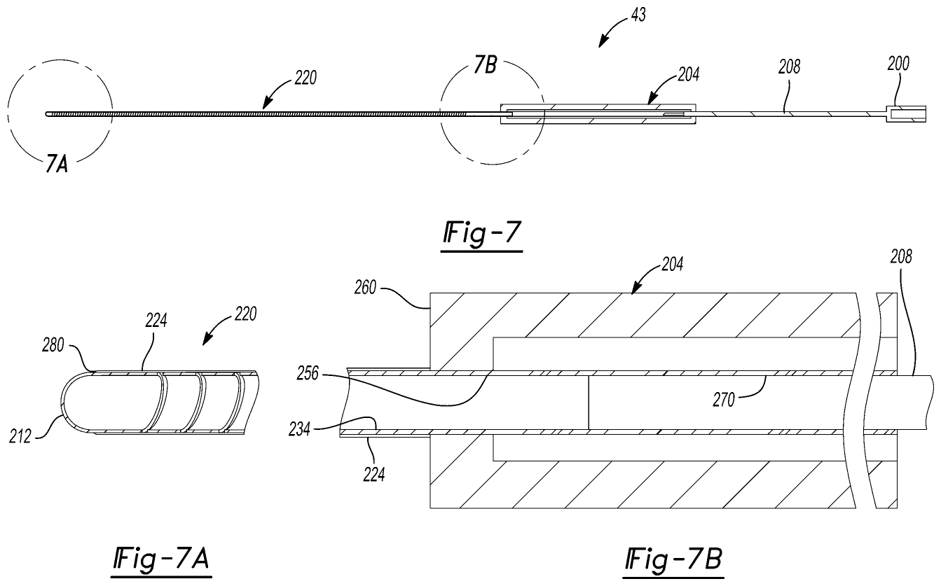

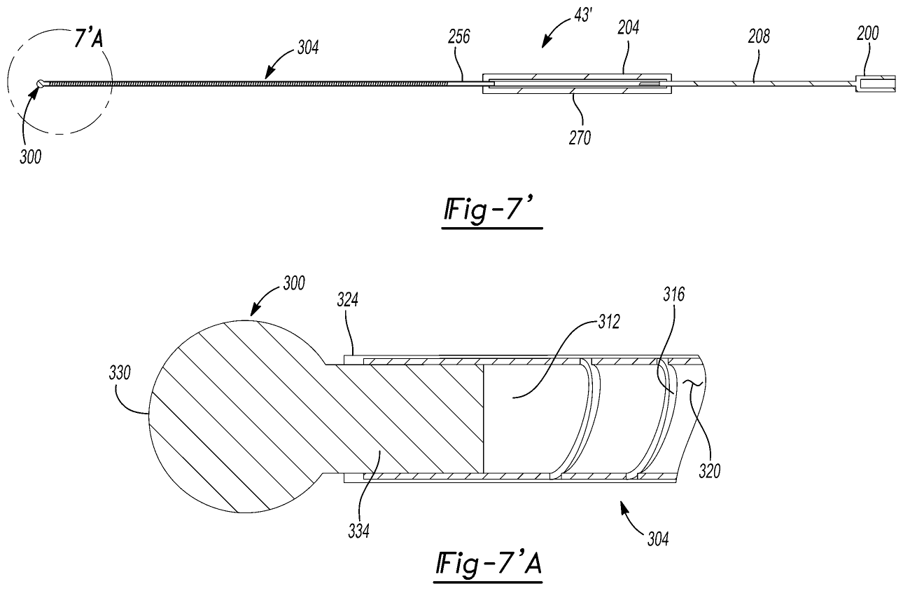

With continuing reference to FIGS. 1-6, and additional reference to FIGS. 7, 7A, and 7B the stimulating probe 43 is further described. As noted above, the flexible number 220 may include the spiral cut 240 formed into the outer wall 234 of the flexible number 220. However, the substantially rigid 256 portion may be uncut. Also, the cut 240 may be a groove or depression formed in the wall 234.

The flexible member 220 may include a distal terminal end that is substantially smooth, curved, and adapted for contact with the subject 36 at the tip 212. The tip 212 may be formed integrally with the flexible member 220 as a single piece. Thus, the tip 212 may carry or conduct a signal, such as an electrical signal, from the connector 200 to the subject 36 and/or from the subject 36 to the connector 200.

As illustrated in detail in FIG. 7B, the handle 204 may include an internal conductive portion 270. The conductive portion 270 may be a separate member, such as a wire or tube. The conductive portion 270 may be formed of a material similar or identical to the flexible member 220. Thus, the conductive portion 270 may be electrically conductive. In various embodiments the conductive portion 270 is formed as a separate member and inserted into the handle and/or formed with the handle 204, such as by molding over the conductive portion 270. Further, the conductive portion 270 may be a coating formed on an interior surface 271 of a bore 273 formed in the handle 204. Moreover, the conductive portion 270 may be the rigid portion 256 that is inserted into the handle 204 to connect directly to the connector 208.

The conductive portion 270 may be in electrical contact with the rigid portion 256 of the flexible number 220. The conductive portion 270 may also be in conductive contact with any other appropriate portion of the flexible member 220, including the portion that includes the spiral cut 240. Thus, an electrical signal may be transferred from the conductive portion 270 to the flexible number 220 and vice versa. The conductive portion 270 may also be connected, such as electrically, to the connector or connector 208. Thus, a signal may be continuously carried from the connector 200 to the tip 212 and vice versa.

To ensure that a selected signal, such as a stimulation signal or an evoked response signal, is selectively carried only from the tip 212, the insulator 234 may be provided on the flexible member 220 from the face 260 of the handle 204 to near or adjacent to the tip 212. The insulation portion 224 may end at any appropriate portion such as at a terminal end 280 of the insulating portion 224. Thus, the tip 212 may be the only exposed (e.g., electrically exposed) portion of the flexible member 220, especially to the subject 36.

The user 31 may, therefore, move the tip 212. As illustrated in FIG. 1, the user 31 may position the tip 212 at any appropriate position relative to the subject 36. The tip 212 may be used to provide a stipulation to a selected portion of the subject 36. The flexible member 220 may allow for a flexibility of the number 220 such that the user 31 may move or position the tip 212 relative to a selected portion of the subject 36. For example, the user 31 may bend or flex the member 220 to position the tip 212 relative to the subject 36. This, the user 31 may curve the member 220 to flex the member 220 around a portion of the subject to position the tip 212. Further the user 31 may apply a force to the tip 212 and the flexible nature of the flexible member 220 may allow for a cushion or reduction of an axial force applied to the subject 36.

With reference to FIGS. 7′ and 7′A, a stimulation probe 43′ is illustrated. The stimulation probe 43′ is similar to the stimulation probe 43 discussed above and similar identical portions will not be described in great detail here. The stimulation probe 43′ may generally include the connector 200, the handle 204, and the connector 208 between the connector 200 and the handle 204. As discussed above, the handle 204 may include an internal conducting member or portion 270 that allows for an electrical connection and conduction between the connector 208 and a tip 300 through a flexible conductor 304. The flexible conductor 304 may include an outer wall 312 that has a spiral cut 316 formed therein. The spiral cut 316 may be substantially similar to the spiral cut 240 of the flexible number 220, thus it may be a complete cut through the wall, a groove, a depression, etc. Therefore, the flexible member 304 may include a structure and operation substantially similar to the flexible number 220, as discussed above. Further, the flexible member 304 may include a bore 320 such that the flexible member 304 resembles a cannula.

The flexible member 304 may be fixed to the handle 204 as discussed above. The flexible member 304 may include a connection or contact with the conductor 270. An insulating portion 324 may be formed on the exterior of the wall 312 to allow for an electrical connection from the tip 300 to the connector 200. With the insulation, the connection may be only between the tip 300 and the connection 200

The tip 300 may be formed as a member that is separate from the flexible member 304. In various embodiments, the tip 300 may be formed of a conductive material, such as a metal, metal alloy, conductive polymer, carbon, etc. As illustrated in FIG. 7′A, the tip 300 include a curved portion 330 and an elongated portion 334. The elongated portion may be fit within the bore 320 of the flexible member 304. The tip 300 may be fixed in the bore 320 in any appropriate manner such as by welding, brazing, conductive adhesive, or the like. The tip 300 may be press fit into the flexible member 304 as well. Regardless, the tip 300 may be formed of a conductive member that allows for an electrical connection to and through the flexible member 304 to the connector 200. Thus, a signal may be passed through the tip 300 either to or from the contractor 200.

According to various embodiments, therefore, the stimulation probe or 43, 43′ may be formed with the flexible portion 220, 304. The flexible portion may allow for ease of positioning of the respective tip 212, 300 relative to the subject 36. The positioning of the respective tip 212, 300 can allow for precise positioning of a stimulation onto or into the subject 36. Further, the flexible portion 220, 304 may allow for an elasticity between the handle 204 and the respective tips 212, 300 to assist in reducing a pressure applied to the subject 36, especially in an actual direction of the stimulation probe 43, 43′.

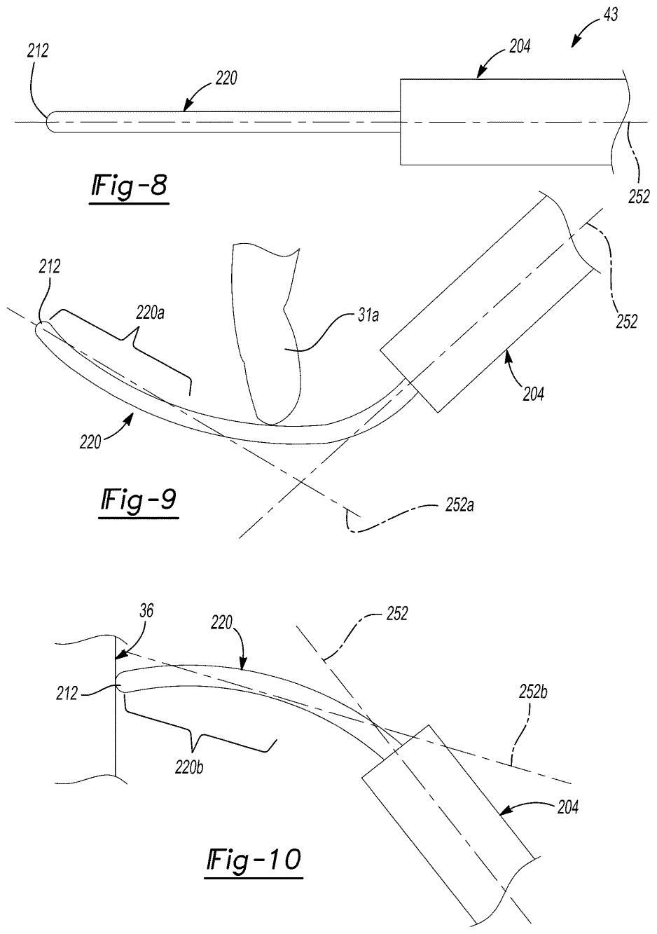

According to various embodiments, and with exemplary reference to the stimulation probe 43 discussed above, the tip 212 may be positioned relative to the handle 204 as illustrated in FIGS. 8, 9, and 10. While the stimulation probe 43 is illustrated as an exemplary stimulation probe in FIGS. 8, 9, and 10, it is understood that the flexible portion or member of the stimulation probe according to various embodiments may include a similar features.

As illustrated in a first configuration, the flexible member 200 may extend along the axis 252. The axis 252 may be defined as long axis of the handle 204. Therefore, the tip 212 may be aligned and positioned on the axis 252 when the flexible member 220 is in the first configuration or position. The first configuration is illustrated in a substantially straight, i.e., un-flexed or un-curved, configuration.

With reference to FIG. 9, the flexible member 220 may be flexed in a second configuration or position such that at least a portion, such as a distal portion 220a extends along an axis 252a that is not aligned with the axis 252 of the handle 204. The flexible member 220 may form a curve that extends along an arc and/or may have a first portion that is straight and a second portion that is curved. Thus, it is understood that to the flexible number 220 may bend relative to the axis 252. The curve or flexing of the flexible number 220 may be encouraged or caused due to a pressure applied by the user 31, such as with a digit 31a. Thus, the user 31 may bend or flex the flexible number 220 in a selected manner.

With reference to FIG. 10, the flexible member 220 may also flex in at least a third configuration. The flexible member 228 may include a curved portion 220b that extends along axis 252b. Again, it is understood that to the flexible number 220 may be curved and at least a portion thereof define an arc. The flexible number 220 may form the curve due to a pressure applied to the tipped 212 as the tip 212 engages or contacts the subject 36. Thus, the user 31 may push the flexible number 220 against the subject 36 to cause the flexible member 220 to deflect and bend.

Accordingly the flexible member 220 may flex in any appropriate configuration, including the configurations illustrated above. The configurations are not numbered to require an order, but only to identify that more than one configuration may occur. Further, the flexible member 220, 304, according to any embodiments, may be elastically flexed. Thus, the flexible member 220, 304 may return to a selected or initial configuration after being flexed, such as straight as illustrated in FIG. 8. It is understood, however, that the flexible member may be configured to maintain a selected configuration such as selecting the insulation material to have a selected memory and/or the material of the flexible member 220, 304. Instruments with curved members include those disclosed in U.S. Pat. No. 10,166,013, incorporated herein by reference. Moreover it is understood that the flexible number 220 may flex in more than one a curve, such as similar to a sinusoidal curve over more than one period. Nevertheless, the flexible member 220 may allow the tip 212 to be positioned such that it is not aligned relative to the axis 252 and/or that the flexible number 220 may form an arc or have at least a portion that is not aligned, such as curved, with the axis 252.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A monitoring system, comprising:
a first portion extending along a first axis;
a second portion having a flexibility greater than the first portion, wherein the second portion is configured to at least in part extend along the first axis in a first configuration and extend along a second axis in a second configuration;
a stimulation portion; and
a stimulation connection to the stimulation portion;
wherein the stimulation portion is configured to be positioned to stimulate a selected portion of a subject.

2. The system of claim 1, further comprising:
a monitoring assembly;
wherein the stimulation connection is configured to provide a stimulation signal form the monitoring assembly to the stimulation portion.

3. The system of claim 1, wherein the second portion is configured to extend along a third axis in a third configuration.

4. The system of claim 1, wherein the second portion is configured to bend upon application of a force by a user.

5. The system of claim 4, wherein the force includes a force applied by a human user against a surface of a human patient.

6. The system of claim 1, wherein is at least one of a wired connection or a wireless connection.

7. The system of claim 1, further comprising:
an insulation layer covering the second portion;
wherein the stimulation portion extends from the second portion beyond the insulation layer.

8. The system of claim 7, wherein the second portion is formed as an elongated member having a bore therein and a spiral cut formed on or through at least a portion of a wall of the second portion;
wherein the insulation layer is configured to bend with the second portion.

9. The system of claim 1, wherein the stimulation portion is at least one of formed as one piece with the second portion or formed as a separate member from, but electrically connected to, the second portion.

10. The system of claim 1, wherein at least the second portion and the stimulation portion are configured to be electrically conductive.

11. A method of operating a monitoring system, comprising:
providing a first portion extending along a first axis;
configuring a second portion to extend from the first portion and have a flexibility greater than the first portion, wherein at least a portion of the second portion is configured to extend along the first axis in a first configuration and extend along a second axis in a second configuration;
providing a stimulation portion electrically connected to the second portion;
forming a stimulation connection with the stimulation portion; and
configuring the stimulation portion to be positioned to stimulate a selected portion of a subject.

12. The method of claim 11, further comprising:
monitoring the subject for an evoked response with a monitoring assembly based on the stimulation from the stimulation portion.

13. The method of claim 11, further comprising configuring the second portion to extend along a third axis in a third configuration.

14. The method of claim 11, further comprising forming the second portion to bend upon application of a force by a user.

15. The method of claim 11, further comprising:
providing an insulation layer covering the second portion; and
placing the stimulation portion to extend from the second portion beyond the insulation layer.

16. The method of claim 15, forming a spiral cut formed on or through at least a portion of a wall of the second portion; and
providing the insulation layer to bend with the second portion.

17. The method of claim 11, further comprising forming the stimulation portion as at least one of one piece with the second portion or as a separate member from, but electrically connected to, the second portion.

18. The method of claim 11, further comprising providing at least the second portion and the stimulation portion to be electrically conductive.
